(19) United States
(12) Patent Application Publication
(10) Pub. No.: US 2004/0266755 A1
(43) Pub. Date: Dec. 30, 2004

(54) Prodrugs of 1-(1-hydroxy-5-isoquinolinesulfonyl) homopiperazine

(75) Inventors: Imadul Islam, Hercules, CA (US); Anthony Johns, Hercules, CA (US); Louis Mylecraine, Wantage, NJ (US); Rene Pagila, Martinez, CA (US); Gary Phillips, Pleasant Hill, CA (US); Babu Subramanyam, Benicia, CA (US); Hans Joachim Zentel, Montvale, NJ (US)

Correspondence Address: BERLEX BIOSCIENCES, PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 10/857,572

(22) Filed: May 28, 2004

Related U.S. Application Data
(60) Provisional application No. 60/474,141, filed on May 29, 2003.

(51) Int. Cl. A61K 31/551; C 07D 4 3/14

(52) U.S. Cl. 514/218; 540/575

(57) ABSTRACT

The present invention provides for novel prodrugs of 1-(1-Hydroxy-5-isoquinolinesulfonyl)homopiperazine of the following Formula I

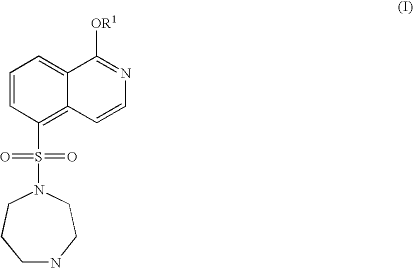

wherein R¹ is as defined herein.

This application claims priority to U.S. Provisional Application Ser. No. 60/474,141 filed May 29, 2003 the entirety of which is incorporated herein by reference.

### BACKGROUND OF THE INVENTION

U.S. Pat. No. 4,678,783 discloses a class of substituted isoquinolinesulfonyl compounds that are useful in treating a variety of disorders including angina. Included in the disclosure of U.S. Pat. No. 4,678,783 is the compound 1-(5-isoquinolinesulfonyl) homopiperazine,

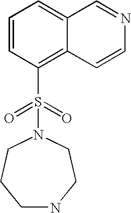

(also known as fasudil, hexahydro-1-(5-isoquinoline-sulfonyl)-1H-1,4,-diazepine, and HA1077) which is a potent rho kinase inhibitor currently in clinical trials as a potential new therapy for stable angina. The entirety of U.S. Pat. No. 4,678,783 is incorporated herein by reference. Further technical advances relating to fasudil are disclosed in U.S. Pat. Nos. 5,942,505 and 6,699,508. The entirety of each of these additional patents is also incorporated herein by reference.

The major active metabolite of fasudil is 1-(1-hydroxy-5-isoquinolinesulfonyl) homopiperazine (or hydroxyfasudil)

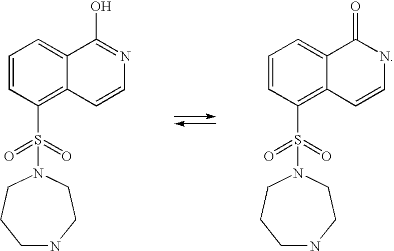

Hydroxyfasudil has a more specific inhibitory effect on Rho-kinase than fasudil (Shimokawa et al. (1999) Cardiovas. Res. 43:1138-1141).

### SUMMARY OF THE INVENTION

We have discovered novel prodrugs of hydroxyfasudil, whose metabolism can lead to in vivo production of the active metabolite hydroxyfasudil. As explained in further detail below, the administration of such prodrugs could provide a potential advantage over the administration of fasudil.

The present invention is directed to compounds of Formula (I)

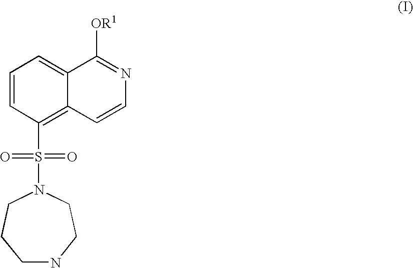

including enantiomers, diastereomers, salts and solvates thereof

wherein

R¹ is

(a) alkyl, cycloalkyl, alkenyl, alkynyl, (cycloalkyl)alkyl, (aryl)alkyl, (heteroaryl)alkyl, (heterocyclo)alkyl, aryl, heteroaryl or heterocyclo any of which may be optionally independently substituted as valence allows with 1 to 3 Z groups; or

(b) —C(═O)R²;

R² is alkyl, cycloalkyl, alkenyl, alkynyl, (cycloalkyl)alkyl, (aryl)alkyl, (heteroaryl)alkyl, (heterocyclo)alkyl, aryl, heteroaryl or heterocyclo any of which may be optionally independently substituted as valence allows with 1 to 3 Z groups; and

Z at each occurrence is independently

(1) V, where V is

(i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;

(ii) a group (i) which is itself substituted by one or more of the same or different groups (i); or

(iii) a group (i) or (ii) which is independently substituted by one or more (preferably 1 to 3) of the following groups (2) to (13),

(2) —OH or —OV,

(3) —SH or —SV,

(4) —C(═O)H, —C(═O)OH, —C(═O)V, —C(═O)OV, or —O—C(O)V,

(5) —SO₃H, —S(O)ₜV, or S(O)ₜN(V¹)V, where t is 1 or 2

(6) halo,

(7) cyano,

(8) nitro,

(9) —U¹—NV²V³,

(10) —U¹—N(V¹)—U²—NV²V³,

(11) —U¹—N(V⁴)—U²—V,

(12) —U¹—N(V⁴)—U²—H,

(13) oxo;

U¹ and U² are each independently

(1) a single bond,

(2) —U³—S(O)ₜ—U⁴—,

(3) —U³—C(O)—U⁴—,

(4) —U³—C(S)—U⁴—,

(5) —U³—O—U⁴—,

(6) —U³—S—U⁴—,

(7) —U³—O—C(O)—U⁴—,

(8) —U³—C(O)—O—U⁴—,

(9) —U³—C(═NV^{1a})—U⁴—, or

(10) —U³—C(O)—C(O)—U⁴—;

V¹, V^{1a}, V², V³ and V⁴

(1) are each independently hydrogen or a group provided in the definition of Z; or

(2) V² and V³ may together be alkylene or alkenylene, completing a 3- to 8-membered saturated or unsaturated ring together with the atoms to which they are attached, which ring is unsubstituted or substituted with one or more groups listed in the definition of Z, or

(3) V² or V³, together with V¹, may be alkylene or alkenylene completing a 3- to 8-membered saturated or unsaturated ring together with the nitrogen atoms to which they are attached, which ring is unsubstituted or substituted with one or more groups listed in the definition of Z; and

U³ and U⁴ are each independently

(1) a single bond,

(2) alkylene,

(3) alkenylene, or

(4) alkynylene.

The present invention is further directed to a pharmaceutical composition comprising a compound of Formula (I) together with a pharmaceutically acceptable vehicle or carrier.

The present invention is further directed to a method of treating a disorder mediated by rho kinase comprising administering to a patient in need thereof an amount of a compound of Formula (I) sufficient to provide in vivo a therapeutically effective amount of hydroxyfasudil.

### DETAILED DESCRIPTION OF INVENTION

The prodrug compounds of Formula (I) may have less potential for in vivo drug-drug interaction as they may not interact with critical cytochrome P450 isoenzymes involved in the metabolism of drugs.

Preferred compounds of Formula I include compounds where R¹ is alkyl, cycloalkyl, (cylcoalkyl)alkyl, (aryl)alkyl, (heteroaryl)alkyl or (heterocyclo)alkyl.

More preferred compounds of Formula I include compounds where R¹ is alkyl.

Most preferred compounds of Formula I include compounds where R¹ is C₁-C₃ alkyl.

The term “alkyl” is used herein at all occurrences (as a group per se or a part of a group) to mean straight or branched chain alkyl groups of 1 to 6 carbon atoms, unless the chain length is otherwise indicated, including, but not limited to methyl, ethyl, n-propyl, isopropyl, n-butyl, sec-butyl, isobutyl, tert-butyl, and the like. Alkyl groups may also be substituted one or more times by halogen, aryl, substituted aryl, hydroxy, methoxy, amino, substituted amino, nitro, carboxy, or cyano.

Where alkyl groups as defined above have single bonds for attachment to two other groups, they are termed “alkylene” groups (e.g., methylene). Similarly, where alkenyl groups as defined above and alkynyl groups as defined above, respectively, have single bonds for attachment to two other groups, they are termed “alkenylene groups” and “alkynylene groups” respectively.

The term “cycloalkyl” as used herein by itself or as part of another group refers to saturated and partially unsaturated (containing 1 or 2 double bonds) cyclic hydrocarbon groups containing 1 to 3 rings, including monocyclicalkyl, bicyclicalkyl and tricyclicalkyl, containing a total of 3 to 20 carbons forming the rings, preferably 3 to 7 carbons, forming the ring. The rings of multi-ring cycloalkyls may be either fused, bridged and/or joined through one or more spiro union to 1 or 2 aromatic, cycloalkyl or heterocyclo rings. Exemplary cycloalkyl groups include cyclopropyl, cyclobutyl, cyclopentyl, cyclohexyl, cycloheptyl, cyclooctyl, cyclodecyl, cyclododecyl, cyclopentenyl, cyclohexenyl, cycloheptenyl, cyclooctenyl, cyclohexadienyl, cycloheptadienyl,

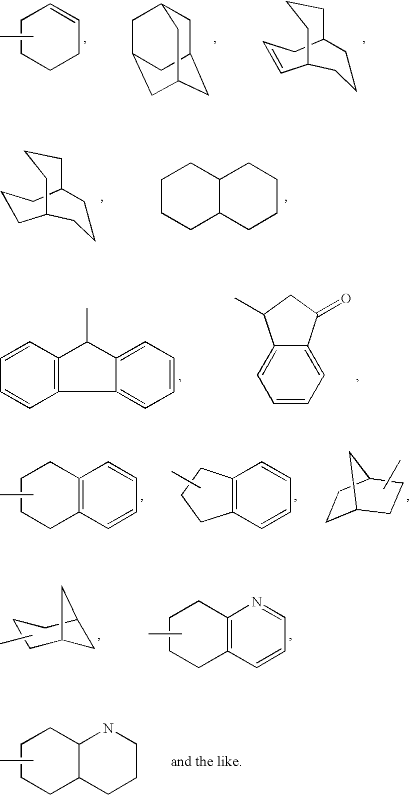

and the like.

“Alkoxy” means alkyl-O— groups in which the alkyl portion (substituted or unsubstituted) is in accordance with the previous definition. Suitable alkoxy groups include methoxy, ethoxy, propoxy and butoxy.

Alkenyl represents C₂-C₆ carbon chains having one or two unsaturated bonds, provided that two unsaturated bonds are not adjacent to each other.

The terms “halo” or “halogen” are used interchangeably herein at all occurrences to mean radicals derived from the elements chlorine, fluorine, iodine or bromine. “Halogenated” is analogous and refers to a degree of halogen substitutions from single to full (per) substitution. The term “haloalkyl” represents a straight or branched alkyl chain substituted by 1 to 5 halo atoms, which can be attached to the same or different carbon atoms, e.g., —CH₂F, —CHF₂, —CF₃, F₃CCH₂— and —CF₂CF₃.

The term “heteroaryl” as used herein by itself or as part of another group refers to monocyclic and bicyclic aromatic rings containing from 5 to 10 atoms, which includes 1 to 4 hetero atoms such as nitrogen, oxygen or sulfur, and such rings fused to an aryl, cycloalkyl, heteroaryl or heterocyclo ring, where the nitrogen and sulfur heteroatoms may optionally be oxidized and the nitrogen heteroatoms may optionally be quaternized. Examples of heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl, indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridyl, dihydroisoindolyl, tetrahydroquinolinyl, carbazolyl, benzidolyl, phenanthrollinyl, acridinyl, phenanthridinyl, xanthenyl

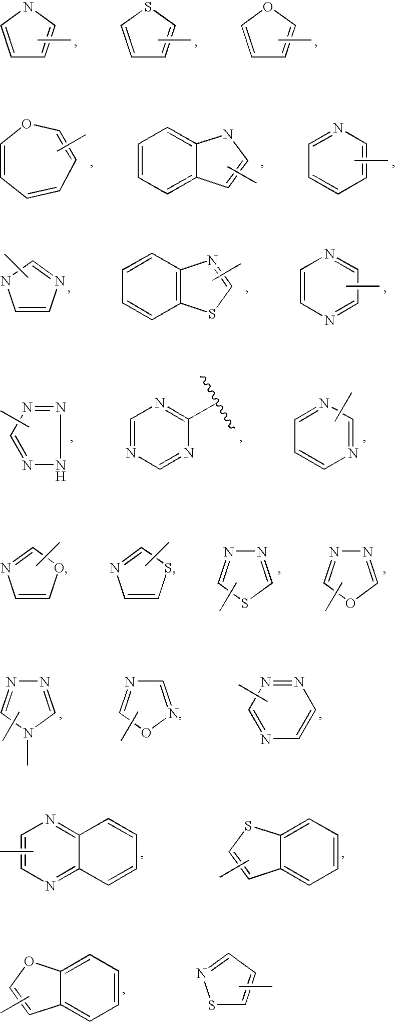

and the like.

The terms “heterocyclic” or “heterocyclo” as used herein by itself or as part of another group refer to optionally substituted, fully saturated or partially unsaturated cyclic groups (for example, 3 to 13 member monocyclic, 7 to 17 member bicyclic, or 10 to 20 member tricyclic ring systems, preferably containing a total of 3 to 10 ring atoms) which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system, where valance allows. The rings of multi-ring heterocycles may be either fused, bridged and/or joined through one or more spiro unions to 1 or 2 aromatic, heteroaryl or cycloalkyl rings. Exemplary heterocyclic groups include azetidinyl, pyrrolidinyl, oxetanyl, imidazolinyl , oxazolidinyl, isoxazolinyl, thiazolidinyl, isothiazolidinyl, tetrahydrofuranyl, piperidinyl, piperazinyl, 2-oxopiperazinyl, 2-oxopiperidinyl, 2-oxopyrrolodinyl, 2-oxoazepinyl, azepinyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl,

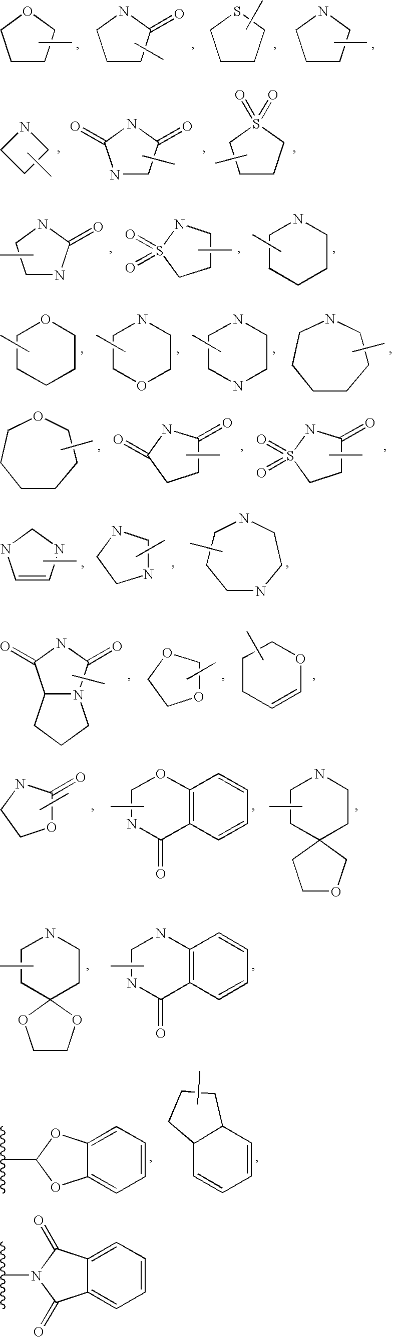

and the like.

The terms “ar” or “aryl” as used herein by itself or as part of another group refer to aromatic homocyclic (i.e., hydrocarbon) monocyclic, bicyclic or tricyclic aromatic groups containing 6 to 14 carbons in the ring portion (such as phenyl, biphenyl, naphthyl (including 1-naphthyl and 2-naphthyl) and antracenyl) and may optionally include one to three additional rings (either cycloalkyl, heterocyclo or heteroaryl) fused thereto. Examples include:

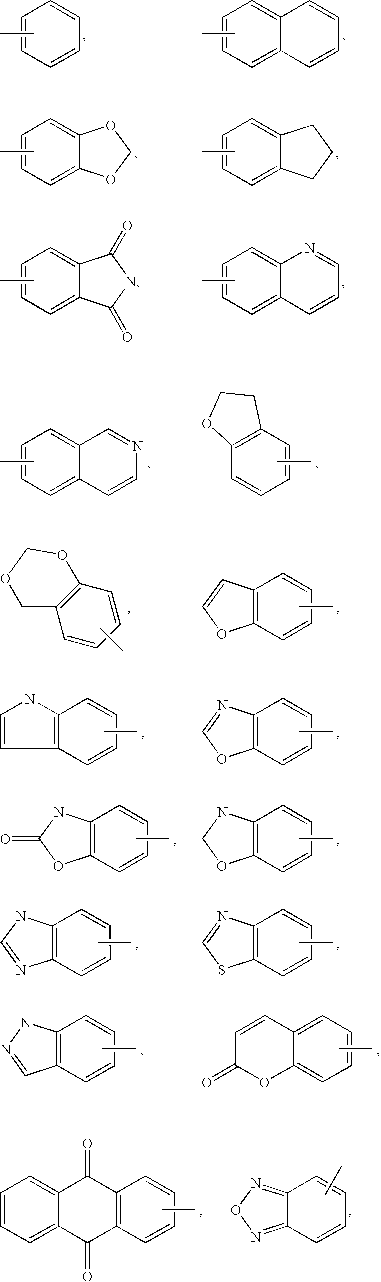

and the like.

The term “arylalkyl”, “aralkyl”, “(aryl)alkyl” or “(ar)alkyl” refers to a residue in which an aryl moiety is attached to the parent structure via an alkyl residue, wherein the aryl and alkyl portions are in accordance with the descriptions above. Similarly, terms such as “(heteroaryl)alkyl”, “(heterocyclo)alkyl”, and “(cycloalkyl)alkyl” refer respectively to heteroaryl, heterocyclo and cycloalkyl moeities that are attached to the parent structure via an alkyl residue.

“Solvates” of the compounds of formula I are preferably hydrates.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

The term “prodrug” is meant to indicate a compound that may be converted under physiological conditions or by solvolysis to hydroxyfasudil. Thus, the term “prodrug” refers to a metabolic precursor of hydroxyfasudil that is pharmaceutically acceptable. A prodrug may be inactive when administered to a subject in need thereof, but is converted in vivo to an active compound of the invention. Prodrugs are typically rapidly transformed in vivo to yield hydroxyfasudil, for example, by hydrolysis in blood. The prodrug compound often offers advantages of solubility, tissue compatibility or delayed release in a mammalian organism (see, Bundgard, H., Design of Prodrugs (1985), pp. 7-9, 21-24 (Elsevier, Amsterdam).

The compounds of the invention are prodrugs of fasudil's major active metabolite, hydroxyfasudil, and thus are useful in treating disorders mediated by rho kinase. Such disorders include hypertension (e.g., systolic hypertension, pulmonary hypertension, essential hypertension, renal hypertension and the like), coronary vasopasm (angina), atherosclerosis, scleroderma, Barter syndrome, transplant atherosclerosis, restenosis, stent stenosis, vein graft stenosis, Reynauds, hypertrophic cardiomyopathy, myocardial infarction, thrombosis, congestive heart failure, aneurysm, cardiac hypertrophy, stroke, cerebral vasopasm (subarachnoid hemorrhage), migraine, spinal cord regeneration, neuronal regeneration, cerebrovascular contraction, cerebrovascular thrombosis, asthma, peripheral circulatory disorders (including, ischemia and intermittent claudication), immature birth, arteriosclerosis, cancer (including bone marrow leukemia, lymphocytic leukemia, gastric cancer, colon cancer, lung cancer, pancreatic cancer, liver cancer, cancer of the esophagus, ovarian cancer, breast cancer, skin cancer, cervical cancer, urinary epithelial cancer, multiple myeloma, uterine cancer, melanoma, tumor invasion and metastasis), inflammation, immune disorders (including allergic disorders, organ transplant rejection and the like), autoimmune disorders (including articular rheumatism, lupus, Sjogren's disease, multiple sclerosis, myasthenia gravis, type I diabetes, endocrine ophthalmopathy, primary biliary cirrhosis, Crohn's disease, glomerulonephritis, sarciodosis, psoriasis, pemphigus, hypoplastic anemia and the like), AIDS, bacterial infection of the digestive tract (e.g., disorders caused by the invasion of Salmonella, sysentery bacillus, intestional pathogenic Escherichia coli and the like), osteoporosis, retinopathy (including angiopathic retinopathy, arteriosclerosis retinopathy, central angiospastic retinopathy, central serous retinopathy, circinate retinopathy, diabetic retinopathy, dysproteinemic retinopathy, hypertensive retinopathy, leukemic retinopathy, lipemic retinpoathy, proliferative retinopathy, renal retinopathy, sickle retinopathy, toxemic retinopathy of pregnancy and the like), Parkinson's disease, Alzheimer's disease, erectile dysfunction, irritable bowel disease, hyperactive bladder, stress incontinence, esophageal spasm, renal and biliary colic, brain function disorders (including psychotic conditions due to cerebral hemorrhage, cerebral thrombus, cerbral embolus, subarachnoid hemorrhage, transient cerebral ischemic stroke, hypertensive encephalopathy, cerebral arteriosclerosis, subdural hematoma, extradural hematoma, cerbral hypoxia, cerebral edema, cerebritis, cerebral tumor, mental disorders, metabolite poisoning, drug poisoning, temporal respiratory arrest and the like as well as the sequelae, decreased attention, hyperactivity, logopathy, delayed mental development, lethe, dementia caused by these disorders). Compounds of the present invention are additionally useful in vascular remodeling, and to inhibit reocclusion of blood vessels after stenting.

Administration of a compound of the invention, as a single stereoisomers, a mixture of stereoisomers, or as a racemic mixture of stereoisomers, or as a pharmaceutically acceptable salt thereof, in pure form or in an appropriate pharmaceutical composition, can be carried out via any of the accepted modes of administration or agents for serving similar utilities. Thus, administration can be, for example, orally, nasally, parenterally, pulmonary, topically, transdermally, or rectally, in the form of solid, semi-solid, lyophilized powder, or liquid dosage forms, such as for example, tablets, suppositories, pills, soft elastic and hard gelatin capsules, powders, solutions, suspensions, aerosols, patches, or the like, preferably in unit dosage forms suitable for simple administration of precise dosages. The compositions will include a conventional pharmaceutical carrier or excipient and a compound of the invention as the/an active agent, and, in addition, may include other medicinal agents, pharmaceutical agents, carriers, adjuvants, etc.

As generally disclosed in U.S. Pat. No. 4,678,783 compounds of the present invention may be administered in an amount from 20 to 300 mg per day for an adult in 2 to 3 administrations, as a single stereoisomer, a mixture of stereoisomers, or as a racemic mixture of stereoisomers, or as a pharmaceutically acceptable salt thereof. The results of recent clinical trials with fasudil suggest that the most preferred dose to effectively treat angina is one that results in plasma concentrations of hydroxyfasudil between about 200 ng/mL to about 1500 ng/mL. The compounds of the invention, or their pharmaceutically acceptable salts, are administered in a therapeutically effective amount which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disease-states; and the host undergoing therapy.

The preferred route of administration is oral, using a convenient daily dosage regimen which can be adjusted according to the degree of severity of the disease-state to be treated. For such oral administration, a pharmaceutically acceptable composition containing a compound(s) of the invention, as a single stereoisomer, a mixture of stereoisomers, or as a racemic mixture of stereoisomers, or as a pharmaceutically acceptable salt thereof, is formed by the incorporation of one or more of the normally employed pharmaceutically acceptable excipient(s), such as, for example, pharmaceutical grades of mannitol, lactose, starch, pregelatinized starch, magnesium stearate, sodium saccharine, talcum, cellulose ether derivatives, glucose, gelatin, sucrose, citrate, propyl gallate, and the like. Such compositions take the form of solutions, suspensions, tablets, pills, capsules, powders, sustained release formulations (such as those described in U.S. Pat. No. 6,699,508) and the like.

Preferably such compositions will take the form of capsule, caplet or tablet and therefore will also contain a diluent such as lactose, sucrose, dicalcium phosphate, and the like; a disintegrant such as croscarmellose sodium or derivatives thereof; a lubricant such as magnesium stearate and the like; and a binder such as a starch, gum acacia, polyvinylpyrrolidone, gelatin, cellulose ether derivatives, and the like.

The compounds of the invention, or their pharmaceutically acceptable salts, may also be formulated into a suppository using, for example, about 0.5% to about 50% active ingredient disposed in a carrier that slowly dissolves within the body, e.g., polyoxyethylene glycols and polyethylene glycols (PEG), e.g., PEG 1000 (96%) and PEG 4000 (4%).

Liquid pharmaceutically administrable compositions can, for example, be prepared by dissolving, dispersing, etc., a compound(s) of the invention as a single stereoisomer, a mixture of stereoisomers, or as a racemic mixture of stereoisomers, or as a pharmaceutically acceptable salt thereof, and optional pharmaceutical acceptable adjuvants in a carrier, such as, for example, water, saline, aqueous dextrose, glycerol, ethanol and the like, to thereby form a solution or suspension.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain a therapeutically effective amount of a compound of the invention, as a single stereoisomer, a mixture of stereoisomers, or as a racemic mixture of stereoisomers; or as a cyclodextrin clathrate thereof, or as a pharmaceutically acceptable salt thereof, for treatment of a disease-state characterized by inflammation in accordance with the teachings of this invention.

The compounds of the present invention may be employed alone or in combination with other suitable therapeutic agents, such as diruetics, anti-hypertensive agents, beta blockers, calcium channel blockers, nitrates, and phosphodiesterase inhibitors (including both cGMP and cAMP). If formulated as a fixed dose, such combination products employ the compounds of this invention within the dosage range described and the other pharmaceutically active agent within its effective dosage range. The above other therapeutic agents, when employed in combination with the compounds of the present invention, may be used, for example, in those amounts indicated in the Physicians' Desk Reference (PDR) or as otherwise determined by one of ordinary skill in the art.

### Schemes

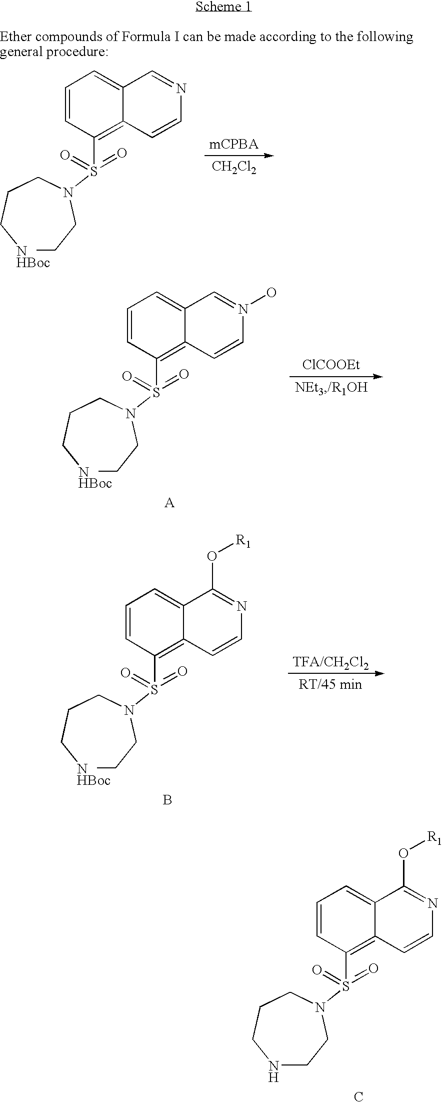

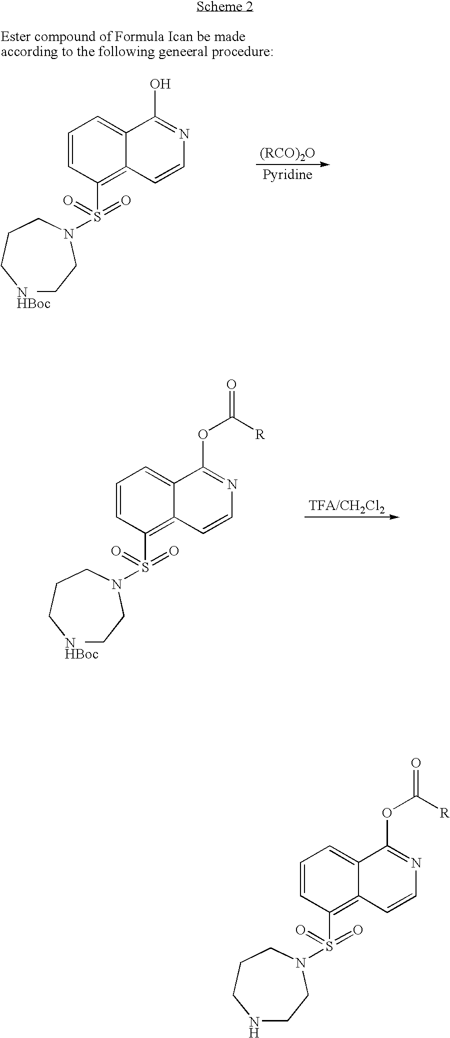

### EXAMPLES

### Example 1

### 1-Ethoxy-5-[(hexhydro-1H-1,4-diazepin-1yl)sufonyl]-isoquinoline.

Preparation of 5-[[4-[(1,1-dimethylethoxy)carbonyl]hexahydro-1H-1,4-diazepin]sufonyl]-2-oxy-isoquinolinium. A

To a solution of Boc-Fasudil (0.82 g, 2.11 mmol) in dichloromethane (50 ml) cooled in ice bath was added m-chloroperbenzoic acid (0.5 g, 2.94 mmol) in portion-wise over a period of 15 min. The reaction mixture was stirred overnight. Solvent evaporated and column chromatography using EtOAc:Methanol (90:10) afforded off-white solid (0.78 g, 91%).

NMR (DMSO-d6)1.38 (s, 9), 1.76 (m, 2H), 3.24-3.38 (m, 4), 3.46 (m, 4), 7.76 (dt, 1), 8.06 (d, 1), 8.14 (d, 1), 8.3 (d, 1), 8.36 (d, 1), 9.1 (s, 1) ppm

1 H-1,4-diazepine-1-carboxylic acid, hexahydro-4-[(1-ethoxy-5-isoquinolinyl)sufonyl]-1,1-dimethylethyl ester: B

To a solution of 5-[[4-[(1,1-dimethylethoxy)carbonyl]hexahydro-1H-1,4-diazepin]sufonyl]-2-oxy-isoquinolinium A (300 mg, 0.73 mmol) in ethanol (5 ml) and ethyl chloro formate (0.1 ml, 1.09 mmol) cooled in an ice bath was triethyl amine (0.20 ml, 1.5 mmol) and the reaction mixture was stirred at room temperature for 14 h. Solvent was evaporated in vacuo and ethyl acetate was added. White solid precipitated out which was discarded. Filtrate was evaporated to and prep HPLC afforded desired ether (130 mg, 80% based on recovered SM) and starting material 150 mg.

NMR (DMSO-d6), 1.40 (s, 9), 1.48 (t, 3), 1.78 (m, 2H), 3.34-3.42 (m, 4), 3.48 (m, 4), 4.58 (q, 2), 7.78 (dt, 1), 7.80 (d, 1), 8.20 (d, 1), 8.30 (d, 1), 8.36 (d, 1), ppm

1-ethoxy-5-[(hexhydro-1H-1,4-diazepin-1yl)sufonyl]-isoquinoline: C

1 H-1,4-diazepine-1-carboxylic acid, hexahydro-4-[(1-ethoxy-5-isoquinolinyl)sufonyl]-1,1-dimethylethyl ester B was dissolved in 10 ml of 20% Triflouroacetic acid in dichloromethane and the reaction mixture was stirred at room temperature for 45 minutes. Solvent was evaporated and reverse phase prep. HPLC afforded 70 mg (50%).

NMR (DMSO-d6), 1.46 (t, 3), 1.94 (m, 2H), 3.14-3.22 (m, 4), 3.42 (t, 2), 3.62 (m, 2), 4.52 (q, 2), 7.78 (t, 1), 7.88 (d, 1), 8.18 (d, 1), 8.28 (dd, 1), 8.52 (d, 1), ppm

Using similar procedures the methyl and isopropyl ether compounds were made.

NMR of Methyl ether (C) and its precursor (B):

1 H-1,4-diazepine-1-carboxylic acid, hexahydro-4-[(1-methoxy-5-isoquinolinyl)sufonyl]-1,1-dimethylethyl ester (B) NMR (CDCl₃), 1.42 (s, 9), 1.94 (m, 2H), 3.34-3.44 (m, 4), 3.48-3.58 (m, 4), 4.16 (s, 3), 7.6 (t, 1), 7.94 (d, 1), 8.16 (d, 1), 8.28 (m, 1), 8.94 (d, 1), ppm

1-Methoxy-5-[(hexhydro-1H-1,4-diazepin-1yl)sufonyl]-isoquinoline (C)

NMR (DMSO-d6), 1.96 (m, 2H), 3.18-3.22 (m, 4), 3.44 (t, 4), 4.08 (s, 3), 7.78 (t, 1), 7.90 (d, 1), 8.20 (d, 1), 8.30 (dd, 1), 8.52 (d, 1), ppm

NMR of Isopropyl ether (C) and its precursor (B):

1 H-1,4-diazepine-1-carboxylic acid, hexahydro-4-[(1-isopropoxy-5-isoquinolinyl)sufonyl]-1,1-dimethylethyl ester (B)

NMR (CDCl₃), 1.48 (s, 9), 1.52 (d, 6), 2.00 (m, 2H), 3.38-3.46 (m, 4), 3.52-3.64 (m, 4), 5.64 (q, 3), 7.62 (t, 1), 7.94 (d, 1), 8.18 (d, 1), 8.34 (m, 1), 8.62 (d, 1), ppm

1-Isopropoxy-5-[(hexhydro-1H-1,4-diazepin-1yl)sufonyl]-isoquinoline (C)

NMR (DMSO-d6), 1.40 (d, 6), 1.98 (m, 2H), 3.18-3.22 (m, 4), 3.44 (t, 2), 3.60 (t, 2), 5.50 (q, 1), 7.76 (t, 1), 7.84 (d, 1), 8.18 (d, 1), 8.28 (dd, 1), 8.48 (d, 1), ppm

For preparation of ethers the following reference procedure was used: Mitsuo Hayashida, Haruyoshi Honda, and Masatomo Hamana, Heterocycles, 1990, 31, 1325-1331.

We claim:

1. A compound of Formula (I) 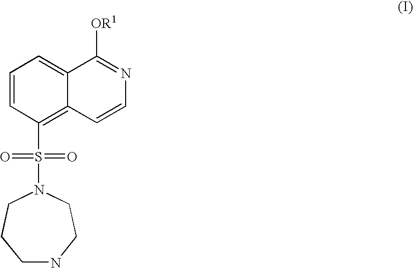
including enantiomers, diastereomers, salts and solvates thereof
wherein
R¹ is
(a) alkyl, cycloalkyl, alkenyl, alkynyl, (cycloalkyl)alkyl, (aryl)alkyl, (heteroaryl)alkyl, (heterocyclo)alkyl, aryl, heteroaryl or heterocyclo any of which may be optionally independently substituted as valence allows with 1 to 3 Z groups; or
(b) —C(═O)R²;
R² is alkyl, cycloalkyl, alkenyl, alkynyl, (cycloalkyl)alkyl, (aryl)alkyl, (heteroaryl)alkyl, (heterocyclo)alkyl, aryl, heteroaryl or heterocyclo any of which may be optionally independently substituted as valence allows with 1 to 3 Z groups; and
Z at each occurrence is independently
(1) V, where V is
(i) alkyl, (hydroxy)alkyl, (alkoxy)alkyl, alkenyl, alkynyl, cycloalkyl, (cycloalkyl)alkyl, cycloalkenyl, (cycloalkenyl)alkyl, aryl, (aryl)alkyl, heterocyclo, (heterocylco)alkyl, heteroaryl, or (heteroaryl)alkyl;
(ii) a group (i) which is itself substituted by one or more of the same or different groups (i); or
(iii) a group (i) or (ii) which is independently substituted by one or more (preferably 1 to 3) of the following groups (2) to (13),
(2) —OH or —OV,
(3) —SH or —SV,
(4) —C(═O)H, —C(═O)OH, —C(═O)V, —C(═O)OV, or —O—C(O)V,
(5) —SO₃H, —S(O)ₜV, or S(O)ₜN(V¹)V, where t is 1 or 2
(6) halo,
(7) cyano,
(8) nitro,
(9) —U¹—NV²V³,
(10) —U¹—N(V¹)—U²—NV²V³,
(11) —U¹—N(V⁴)—U²—V,
(12) —U¹—N(V⁴)—U²—H,
(13) oxo;
U¹ and U² are each independently
(1) a single bond,
(2) —U³—S(O)ₜ—U⁴—,
(3) —U³—C(O)—U⁴—,
(4) —U³—C(S)—U⁴—,
(5) —U³—O—U⁴—,
(6) —U³—S—U⁴—,
(7) —U³—O—C(O)—U⁴—,
(8) —U³—C(O)—O—U⁴—,
(9) —U³—C(═NV^{1a})—U⁴—, or
(10) —U³—C(O)—C(O)—U⁴—;
V¹V^{1a}V²V³ and V⁴
(1) are each independently hydrogen or a group provided in the definition of Z; or
(2) V² and V³ may together be alkylene or alkenylene, completing a 3- to 8-membered saturated or unsaturated ring together with the atoms to which they are attached, which ring is unsubstituted or substituted with one or more groups listed in the definition of Z, or
(3) V² or V³, together with V¹, may be alkylene or alkenylene completing a 3- to 8-membered saturated or unsaturated ring together with the nitrogen atoms to which they are attached, which ring is unsubstituted or substituted with one or more groups listed in the definition of Z; and
U³ and U⁴ are each independently
(1) a single bond,
(2) alkylene,
(3) alkenylene, or
(5) alkynylene.

2. A compound of claim 1 wherein R¹ is —C(═O)R².

3. A compound of claim 1 wherein R¹ is alkyl optionally independently substituted as valance allows with one to three Z groups.

4. A compound of claim 3 wherein R¹ is C₁-C₃ alkyl.

5. A compound of claim 4 selected from
1-Ethoxy-5-[(hexhydro-1H-1,4-diazepin-1yl)sufonyl]-isoquinoline;
1-Methoxy-5-[(hexhydro-1H-1,4-diazepin-1yl)sufonyl]-isoquinoline; and
1-Isopropoxy-5-[(hexhydro-1H-1,4-diazepin-1yl)sufonyl]-isoquinoline including salts and solvates thereof.

6. A pharmaceutical composition comprising at least one compound of claim 1 together with a pharmaceutically acceptable vehicle or carrier.

7. A pharmaceutical composition of claim 6 wherein the compound of formula I is a compound where R¹ is —C(═O)R².

8. A pharmaceutical composition of claim 6 wherein the compound of formula I is a compound where R¹ is alkyl optionally independently substituted as valance allows with one to three Z groups.

9. A pharmaceutical composition of claim 8 wherein R¹ is C₁-C₃ alkyl.

10. A pharmaceutical composition of claim 9 wherein the compound of formula I is selected from
1-Ethoxy-5-[(hexhydro-1H-1,4-diazepin-1yl)sufonyl]-isoquinoline;
1-Methoxy-5-[(hexhydro-1H-1,4-diazepin-1yl)sufonyl]-isoquinoline; and
1-Isopropoxy-5-[(hexhydro-1H-1,4-diazepin-1yl)sufonyl]-isoquinoline including salts and solvates thereof.

11. A method of treating a disorder mediated by rho kinase comprising administering to a patient in need thereof an amount of a compound of claim 1 sufficient to provide in vivo a therapeutically effective amount of hydroxyfasudil.

12. A method of claim 11 where the compound of claim 1 is a compound where R¹ is —C(═O)R².

13. A method of claim 11 where the compound of claim 1 is a compound where R¹ is alkyl optionally independently substituted as valance allows with one to three Z groups.

14. A method of claim 13 where R¹ is C₁-C₃ alkyl.

15. A method of claim 14 where the compound of formula I is selected from
1-Ethoxy-5-[(hexhydro-1H-1,4-diazepin-1yl)sufonyl]-isoquinoline;
1-Methoxy-5-[(hexhydro-1H-1,4-diazepin-1yl)sufonyl]-isoquinoline; and
1-Isopropoxy-5-[(hexhydro-1H-1,4-diazepin-1yl)sufonyl]-isoquinoline including salts and solvates thereof.

16. A method of claim 11 wherein the disorder mediated by rho kinase is selected from hypertension, angina, atherosclerosis, scleroderma, Barter syndrome, transplant atherosclerosis, restenosis, stent stenosis, vein graft stenosis, Reynauds, hypertrophic cardiomyopathy, myocardial infarction, thrombosis, congestive heart failure, aneurysm, cardiac hypertrophy, stroke, subarachnoid hemorrhage, migraine, spinal cord regeneration, neuronal regeneration, cerebrovascular contraction, cerebrovascular thrombosis, asthma, peripheral circulatory disorders, immature birth, arteriosclerosis, cancer, inflammation, immune disorders, autoimmune disorders, AIDS, bacterial infection of the digestive tract, osteoporosis, retinopathy, Parkinson's disease, Alzheimer's disease, erectile dysfunction, irritable bowel disease, hyperactive bladder, stress incontinence, esophageal spasm, renal and biliary colic, and brain function disorders.

17. A method of inhibiting reocclusion of blood vessels after stenting, comprising administering to a patient in need thereof a sufficient amount of a compound of claim 1 to provide in vivo an effective amount of hydroxyfasudil.

18. A pharmaceutical composition of claim 6 further comprising at least one additional therapeutic agent selected from diruetics, anti-hypertensive agents, beta blockers, calcium channel blockers, nitrates, and phosphodiesterase inhibitors.

19. A method of claim 11 further comprising the administration of an effective amount of at least one additional therapeutic agent selected from diruetics, anti-hypertensive agents, beta blockers, calcium channel blockers, nitrates, and phosphodiesterase inhibitors.
